(19) United States
(12) Patent Application Publication
(10) Pub. No.: US 2006/0105339 A1
(43) Pub. Date: May 18, 2006

(54) Methods for measuring the rates of replication and death of microbial infectious agents in an infected

(75) Inventor: Mark K Hellerstein, Kensington, CA (US)

Correspondence Address: MORRISON & FOERSTER LLP, 425 MARKET STREET, SAN FRANCISCO, CA, 94105-2482, US

(73) Assignee: MARC C HELLERSTEIN

(21) Appl. No.: 10/526,860

(22) PCT Filed: Sep. 4, 2003
(86) PCT No.: PCT/US03/27623

§ 371 Date: Oct. 19, 2005

Related U.S. Application Data
(60) Provisional application No. 60/408,346, filed on Sep. 4, 2002.

(51) Int. Cl. C12Q 1/70; C12Q 1/68

(52) U.S. Cl. 435/6; 435/5

(57) ABSTRACT

The present invention provides methods and kits useful for determining rates of replication and destruction of an infectious agent within an infected host organism. In the methods of the invention, an isotopically-labeled precursor molecule is administered to an infected host, and is given sufficient time to pass into the host's metabolic pools into a biochemical component of the infectious agent. The isotopic content and/or pattern or the rate of change of the isotopic content and/or pattern of the biochemical component is then measured to determine the rate of replication (growth) of the infectious organism while in the host. Alternatively, isotopic decay of labeled molecular components of the infectious agent is measured over time after discontinuing administration of the isotopically labeled precursor molecule to determine the rate of destruction (death) of the infectious agent while in the host. Thus, using methods of the invention, in vivo sensitivity of infectious agents to drug agents may be determined, in order to optimize therapy of the infected host.

### CROSS-REFERENCE TO RELATED APPLICATIONS

This application claims priority to U.S. Provisional Patent Application No. 60/408,346 filed on Sep. 4, 2002, which is hereby incorporated by reference in its entirety.

### FIELD OF THE INVENTION

The present invention relates to methods for determining rates of replication and death of infectious microbial agents, such as viruses, bacteria, protozoa, or parasites, while they are present in an infected host organism. In the methods of this invention, isotopically labeled precursor molecules are administered to an infected host to determine the rates of biosynthesis or destruction of an infectious agent in the host.

### BACKGROUND OF THE INVENTION

In the medical field of infectious diseases, the goal of most therapeutic interventions is to alter the rate of proliferation (growth) or destruction (death) of infectious agents, such as viruses, bacteria, protozoa or parasites, during their invasion of a host organism. The efficacy of an antibiotic, antiviral therapeutic, or immunostimulatory treatment is determined by its effect on the growth or death of the infectious agent. Although such effects on infectious agents are usually discussed by authorities in terms of microbiology (i.e., those features of an infectious agent predisposing toward sensitivity or resistance to a therapy), immunology (i.e., those features of a host predisposing toward growth or death of the foreign organism), or molecular cell biology (i.e., the biochemical events involved in replication or death of the infectious organism), from a purely biochemical point of view, it is the replication and destruction of the molecular components of the microbial organism that represent the most basic events. The biochemical component molecules which constitute an infectious agent are synthesized in the process of producing new infectious organisms, or are broken down during the process of destruction (death) of the infectious organisms. The relative rates of these two processes are responsible for the ultimate clinical outcome (i.e., persistence, advancement, or resolution of the infection in the host).

In the field of infectious diseases, the biosynthesis or destruction of component molecules of infectious agents while in the host has not previously been directly measured. Although replication of the biochemical components of an invading microbe is generally appreciated to occur within an organism, these processes have not previously been considered as products of the organism and therefore measurable by classical procedures including endogenous labeling from the host's nutrient pools. Instead, infectious agents have typically been isolated and cultured ex vivo (outside of the host organism), and then exposed to antimicrobial drugs of interest, to determine sensitivities to various drug treatments. Accordingly, therapeutic decisions are often guided by ex vivo drug sensitivities of the infectious organism.

The above approach, however, is not ideal for several reasons. First, some infectious organisms, in particular viruses such as human immunodeficiency virus or other agents that require specific cell types in the host organism for their life cycle, cannot be reliably, relevantly, or usefully cultured ex vivo. Second, the interaction between host defenses (e.g., antibodies, T-cells, cytokines, blood flow) and drugs is not tested by this ex vivo approach, although these factors may be the most important features responsible for successful recovery from an infection. Third, antibiotic concentrations actually achieved in the body tissue of interest may not be the same as those tested ex vivo. Finally, ex vivo measurements can never provide a definitive answer to the ultimate clinical question at hand, i.e., whether an infectious agent is actually growing or being destroyed in a host organism receiving a particular treatment regimen.

Thus, there is a tremendous need for an improved method of measuring proliferation or destruction of an infectious agent within a host organism, particularly in response to a therapeutic regimen.

### BRIEF SUMMARY OF THE INVENTION

In order to meet these needs, the present invention is directed to methods of determining the rate of replication or destruction of an infectious agent in a host organism, by administering an isotope-labeled precursor molecule to the host organism for a period of time sufficient for the label of the isotope-labeled precursor molecule to become incorporated into a biochemical component of the infectious agent; obtaining one or more biological samples from the host organism, wherein the one or more biological samples include an infectious agent or the biochemical component of the infection agent; measuring the isotopic content, and/or pattern or rate of change of isotopic content and/or pattern in the biochemical component; and calculating the rate of synthesis or breakdown of the biochemical component to determine the rate of replication or destruction of the infectious agent in the host organism.

In one aspect, the biological sample may be a tissue of the host organism. The biological sample may be a bodily fluid of the host organism. The biological fluid may be one or more of urine, blood, saliva, interstitial fluid, edema fluid, lacrimal fluid, inflammatory exudates, synovial fluid, abscess, empyema, cerebrospinal fluid, sweat, pulmonary secretions, seminal fluid, feces, bile, and intestinal secretions.

In another aspect, the host organism may be a mammal. The mammal may be a human.

The infectious agent may be one or more of bacteria, viruses, protozoa, yeast, and parasites.

In a further aspect, the infectious agent may be human immunodeficiency virus, hepatitis B or C virus, or other clinically important viruses. Examples of clinically important viruses may be found, for example, in G. Kobayashi, Patrick R. Murray, Michael A. Pfaller, and Ken S. Rosenthal; Medical Microbiology, published by Mosby; 4th edition (Jan. 15, 2002), incorporated herein by reference in its entirety.

The biochemical component is selected from the group consisting of DNA, RNA, proteins, lipids, carbohydrates, and porphyrins.

The isotopic label may be one or more of ¹³C, ¹⁴C, ²H, ³H, ¹⁵N, ³⁵S, ¹¹C, and ³⁵P.

One or more biological samples may be collected from the host organism. In one variation, a plurality of biological samples may be collected from the host organism.

In another aspect, measurement of the isotopic content and/or pattern or the rate of change of isotopic content and/or pattern in the biochemical component is performed by mass spectrometry.

In another variation, the present invention includes a method of identifying an antimicrobial or immunostimulatory effect of a drug agent by determining the rate of replication or destruction of an infectious agent in a host organism; administering the drug agent to the host organism; and determining the rate of replication or destruction of the infectious agent in a host organism, wherein a decrease in the rate of replication or an increase in the rate of destruction of the infectious agent indicates an antimicrobial or immunostimulatory effect of the drug agent. In another variation, the method of identifying an antimicrobial or immunostimulatory effect of a drug agent includes determining the rate of replication or destruction of an infectious agent in a first host organism, wherein a drug agent has not been administered to the first host organism; determining the rate of replication or destruction of an infectious agent in a second host organism, wherein a drug agent has been administered to the second host organism; and comparing the rate of replication or destruction of the infectious agent in the first and second host organisms, wherein a decrease in the rate of replication or an increase in the rate of destruction of the infectious agent in the second host organism indicates an antimicrobial or immunostimulatory effect of the drug agent. Alternatively, the effect of the antimicrobial or immunostimulatory agent on the growth or death of the infectious agent in the host organism may be used as a diagnostic test in clinical patient care or as a biomarker tool for drug discovery, development, or approval of an antimicrobial or immunostimulatory agent.

In another aspect, the present invention includes kit for determining the rate of replication or destruction of an infectious agent in a host organism. The kits include at least one isotope-labeled precursor molecule. The kit may further include instructions for use of the kit, a tool for administration of precursor molecules, and/or an instrument for collecting a sample from a host organism.

In another aspect, the present invention includes the rights to drug agents discovered in the above methods, isolated isotope-labeled precursor molecules, isolated isotopically labeled biochemical components, and isolated infectious agents having one or more isotope labeled biochemical components.

### DETAILED DESCRIPTION OF THE INVENTION

The invention provides, inter alia, methods and kits for assessing rates of replication (growth) or destruction (death) of a microbial infectious agent in a host organism. Methods and kits of the invention may be used for determining virulence of an infectious agent in vivo, and in vivo sensitivity of an infectious agent to an antimicrobial therapeutic regimen, i.e., treatment with a drug agent.

Replication of an invading infectious agent is a biochemical process involving the synthesis of the biochemical components that include the infectious agent from precursor molecules, and that destruction (death) of an invading infectious agent is also a biochemical process involving the breakdown or degradation of molecules that include the infectious organism (e.g., proteins, nucleic acids, lipids and carbohydrates). Moreover, the precursor molecules used for the synthesis of molecules that include the infectious organism are derived from and provided by biochemical pools in the infected host organism (e.g., amino acids, glucose, nucleotides and other biological molecules in the tissues, bloodstream, or other bodily fluids of the host organism). The rates of biosynthetic and degradative processes can be measured by the use of isotope labeling techniques.

Previously, the biosynthesis rates of endogenous molecules in the tissues of living organisms has been measured by administering isotopically-labeled biochemical subunits that enter into the host organism's biochemical and nutrient pools then are incorporated into molecules of interest (see, e.g., U.S. Pat. Nos. 5,338,686, 5,910,403, 6,010,846, and 6,461,806, and U.S. patent application Ser. Nos. 10/366,125 and 10/279,399, which are hereby incorporated by reference in their entirety).

In one embodiment of the present invention, isotopically-labeled precursor molecules are administered to a host organism to determine the synthesis and/or degradation rates of biochemical components of an infectious agent while it invades and/or resides in the host organism. Methods of isotope kinetics, previously only applied to synthesis of endogenous molecules, are herein applied to foreign infectious agents that has invaded a host. The objectives are to measure directly the growth or destruction rates of the infectious agent in the host organism as a diagnostic tool to optimize medical therapy (for example, against the human immunodeficiency virus or multi-drug resistant bacteria). Previously-available methods for measuring microbial replication rates (e.g., U.S. Pat. No. 5,338,686) have not included administration of an isotope labeled precursor molecule to a host organism to measure incorporation of labeled isotopes into a biochemical component of an invading microbe. Methods of the invention thus allow an in vivo assessment of microbial growth that was not available using previous methods.

In an aspect of this invention, an isotopically labeled precursor molecule is administered to a host organism, infected by an infectious agent. The precursor is incorporated from the nutrient metabolic pools of the host into a biochemical component of the infectious agent. A sample of the microbial population is then directly isolated from the host, and the incorporation of isotope label precursor molecules into the biochemical component of the infectious agent is then measured and used to calculate a rate of synthesis of the component molecule using methods well known in the art. The rate of synthesis represents or correlates with the rate of proliferation of the infectious agent in the host organism. The isotopic decay in the component molecule of the infectious agent may also be measured after discontinuing administration of an isotopically labeled precursor molecule to the infected host organism, to calculate the rate of breakdown of the biochemical component, thus indicating the rate of destruction of the infectious agent in the host.

Thus, in one aspect, infectious microbial agents are considered as though they are biochemical products synthesized by a host organism rather than independent, foreign objects, rendering such parameters as pathogenicity, virulence, and therapeutic response measurable in a living subject using metabolic labeling techniques previously developed for the measurement of synthesis and breakdown rates of endogenous biochemical components of an organism (e.g., U.S. Pat. Nos. 5,338,686, 5,910,403, 6,010,846, and 6,461,806, supra). Medical applications of the present invention include determination of the efficacy of antiviral therapeutics, antibiotics, or other treatments and establishment of in vivo sensitivity and dose-response relationships of an infectious agent to chemical compounds such as drugs. Further medical applications include the ability to monitor infectious disease progression in a host organism infected with an infectious agent by determining whether the infectious agent is replicating, remaining static, or is being destroyed, for example by the host organism's immune system. Monitoring of disease progression can be conducted in conjunction with evaluating treatment efficacy, or it can be done independent of any therapeutic intervention.

### ADVANTAGES OF THE INVENTION

Aspects of the invention differ from previous methods in the field of infectious diseases in several fundamental respects:

(1) Direct measurement of microbial processes of interest in vivo. Even though the pathogenesis of infectious diseases involves growth, maintenance, self-replication, and death of microbial organisms in a host organism, previous methods have not actually measured these biochemical and molecular processes or their molecular flux rates in vivo in the host organism.

(2) Direct measurement of antibiotic action on microbes in vivo in the context of host factors. Although the goal of treatment with antibiotics (i.e., antimicrobial pharmaceuticals) is to inhibit the growth, maintenance, and self-replication of microbes or to accelerate the death of microbes in a host organism, previous methods have not measured the direct effects of antibiotics on these biochemical and molecular processes in vivo. Previous methods have utilized ex vivo microbiology techniques, which are independent of and unable to account for host defense factors of the infected organism. In contrast, methods of the present invention may be used to assess the role of host defense factors of the infected organism in microbial virulence or sensitivity to therapeutic intervention and interactions between host factors and microbial factors.

(3) Avoidance of artifacts and limitations related to growth of microbes in an ex vivo culture system. Methods of the invention obviate the need for ex vivo culturing of infectious agents. Ex vivo culture techniques are powerful tools for identifying the presence of a specific microbe, but are fundamentally limited and unreliable for assessing actual infectious activity of a microbe in a living host. For example, viruses are often difficult to grow and require non-physiologic (i.e., artificial) culture conditions. Other classes of microbes that are difficult to culture ex vivo include intracellular infectious agents such as Mycobacterium tuberculosis. The level of infectious activity, virulence, control by antibiotics, and other factors of such infectious agents is difficult to characterize in the clinical setting. Further, localized infections in a host (e.g., abscesses and empyemas) are often not susceptible to penetration by antibiotics in vivo. Accordingly, ex vivo culture and sensitivity tests do not reflect actual in vivo efficacy of a particular therapy, as is widely recognized by clinical infectious disease practitioners. In addition, a compromised host (e.g., a patient undergoing immunosuppressive therapy or a patient with a condition such as AIDS or diabetes mellitus) may not be able to provide the cofactors required to translate ex vivo antibiotic sensitivity into clinical eradication of an invading microbe. Therefore, ex vivo sensitivity of a microbial agent to a treatment does not reliably indicate actual in vivo effectiveness of the treatment in the infected host organism.

(4) Use of the host organism's biosynthetic building blocks to label the infectious agent. Invading infectious agents must use the nutrients and metabolic building blocks provided by a host organism, even though invading infectious agents are independent organisms that are foreign to the host. Therefore, administration of labeled isotope precursor molecules to an infected host organism is equivalent to direct administration of the labeled precursors to the infectious agent itself. Although isotopic labeling techniques have been used previously for measuring the biosynthesis or breakdown rates of endogenous macromolecules, including DNA, in the cells of an organism, such techniques have not been used previously for labeling and measuring the biosynthesis or breakdown rates of macromolecules in an invading microbe. In methods of this invention, isotopically labeled biosynthetic precursors are administered to one organism, an infected host, in order to measure biosynthetic or breakdown rates in another organism, an infectious agent.

General Techniques

The practice of the present invention will employ, unless otherwise indicated, conventional techniques of biochemistry, microbiology, cell biology, and mass isotopomer analysis, which are within the skill of the art. Such techniques are explained fully in the literature, such as, Methods in Enzymology (Academic Press, Inc.); Cell Biology: A Laboratory Notebook (J. E. Cellis, ed., 1998) Academic Press; Mass isotopomer distribution analysis at eight years: theoretical, analytic and experimental considerations (Hellerstein and Neese, Am J Physiol 276 (Endocrinol Metab 39) E1146-E1162, 1999); Biemann, K (1990), Mass spectrometry of Biological Materials, C. McEwen, ed., Marcel Dekker, NY; Wolfe, R. R. (1984), Radio-isotope and stable isotope/mass spectrometric methods, Alan R. Liss, Inc., NY.

Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

As used herein, “precursor molecule” and “biochemical precursor” are used interchangeably to refer to any molecule utilized in one or more specific biochemical pathways to produce a biochemical component of an infectious agent. Precursor molecules and biochemical precursors may contain isotope labels. Examples of isotope-labeled precursor molecules and isotopically labeled biochemical precursors include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃. Examples of precursors suitable for use in the methods of the invention include isotopically-labeled amino acids, fatty acids, carbohydrates, purine or pyrimidine bases, CO₂, H₂O, and NH₃.

“Biochemical component” refers to a constituent part of an infectious agent that is synthesized from precursor molecules. Typically, a biochemical component is a “biopolymer” or “macromolecule,” a molecule that is synthesized in a biological system using discrete subunits as precursors. Examples of biochemical components include, but are not limited to, amino acids, carbohydrates, fatty acids, peptides, sugars, lipids, nucleic acids, polynucleotides, glycosaminoglycans, polypeptides, proteins, and combinations thereof that are present within a metabolic pathway within a living system.

“Infectious agent,” “microbial infectious agent,” “invading microbe,” and “microbe” are used interchangeably, and refer to any entity capable of infecting a host organism and that exhibits recognized features of living systems, including but not limited to the capacity for self-replication and generation of off-spring, the presence of nucleic acids as the genetic material, and the presence of organic molecules synthesized from smaller subunits. Examples of infectious agents include, but are not limited to bacteria, viruses, protozoa, yeast, and parasites, and any organism capable of replicating in a host organism, whether extracellularly, intracellularly, or both. See, e.g., G. Kobayashi, Patrick R. Murray, Michael A. Pfaller, and Ken S. Rosenthal; Medical Microbiology, published by Mosby; 4th edition (Jan. 15, 2002), incorporated herein by reference in its entirety.

As used herein, “parasite” refers to any organism which lives in, on, or with another organism (i.e., a host organism) and obtains nutrients from the host organism. As used herein, a parasite may provide benefit or no benefit to the host organism. Examples of parasites include, but are not limited to, viruses, bacteria, protozoa, and yeast, as well as multicellular organisms such as helminthes, pinworms, giardia and the like. The term also includes many unicellular protozoa (such as species of the genus plasmodium, various species of amoeba, and the like) that inhabit host organisms.

As used herein, “clinically important infectious agent” is an infectious agent, microbial infectious agent, invading microbe, microbe, virus, bacteria, protozoa, yeast, or parasite that causes or is associated with a disease or disorder in an individual. See, e.g., G. Kobayashi, Patrick R. Murray, Michael A. Pfaller, and Ken S. Rosenthal; Medical Microbiology, published by Mosby; 4th edition (Jan. 15, 2002), incorporated herein by reference in its entirety.

“Replication,” “growth,” and “proliferation” of an infectious agent are used interchangeably and refer to an increase in the number of microbes in the host organism.

“Death” and “destruction” of an infectious agent are used interchangeably to refer to a decrease in the number of microbes in the host organism. The decrease in number of microbes is the result of the irreversible loss of viability or capacity for self-maintenance or replication.

As used herein, a “host” or “host organism” is an organism in which an infectious agent is capable of replicating. An “infected host” or “infected host organism” is a host containing an infectious agent. The host may be any organism, preferably an animal, more preferably a vertebrate, most preferably a mammal. Examples of mammals include humans, nonhuman primates, farm animals, pet animals, for example cats and dogs, and research animals, for example mice and rats. As used herein, the terms “host” and “host organism” may also be a cell grown in culture, such as, for example, a mammalian tissue culture cell.

“Molecular flux rates” refers to the rate of synthesis and/or breakdown of molecules within a cell, tissue, or organism. “Molecular flux rates” also refers to a molecule's input into or removal from a pool of molecules, and is therefore synonymous with the flow into and out of the pool of molecules.

“Metabolic pathway” refers to any linked series of two or more biochemical steps in a living system, the net result of which is a chemical, spatial or physical transformation of a molecule or molecules. Metabolic pathways are defined by the direction and flow of molecules through the biochemical steps that include the pathway. Molecules within metabolic pathways can be of any biochemical class, including but not limited to lipids, proteins, amino acids, carbohydrates, nucleic acids, polynucleotides, porphyrins, glycosaminoglycans, glycolipids, intermediary metabolites, inorganic minerals, and ions.

“Flux rate through a metabolic pathway” refers to the rate of molecular transformations through a defined metabolic pathway. The unit of flux rates through pathways is chemical mass per time (e.g., moles per minute, grams per hour). In one aspect, the flux rate through a pathway refers to the transformation rate from a clearly defined biochemical starting point to a clearly defined biochemical end-point, including all the stages in between in the defined metabolic pathway of interest.

“Isotopes” refer to atoms with the same number of protons and hence of the same element but with different numbers of neutrons (e.g., ¹H vs. ²H vs. ³H). The chemical symbol for deuterium, ²H, is commonly represented as “D,” as is well known in the art.

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition, therefore each isotopologue has a unique exact mass but may not have a unique structure. An isotopologue is usually comprised of a family of isotopic isomers (isotopomers) which differ by the location of the isotopes on the molecule (e.g., CH₃NHD and CH₂DNH₂ are the same isotopologue but are different isotopomers).

“Isotope-labeled water” includes water labeled with one or more specific heavy isotopes of either hydrogen or oxygen. Specific examples of isotope-labeled water include ²H₂O,³H₂O, and H₂¹⁸O.

“Isotope content” refers to the total amount or content of an incorporated isotope present in the biochemical component molecule or to the proportion of a particular isotopic species of the biochemical component molecule that is present.

“Isotopic pattern” refers to the relative values of, or quantitative relationships among, isotopic species of the biochemical component molecule, e.g., the relative proportions of different mass isotopomers of the molecule.

As used herein, “drug agent” refers to any chemical entity, known drug or therapy, potential drug or therapy, approved drug or therapy, antimicrobial agent, antiviral agent, antiparastic agent, or antiprotozoal agent, or any biological agent including, but not limited to, a nucleotide sequence such as for gene therapy, a monoclonal antibody, antibiotic, or immunostimulatory agent such as an interleukin.

“Living system” or “host organism” or “living organism” include, but are not limited to, cells, cell lines, plants, animals, mammals, guinea pigs, rabbits, dogs, cats, domesticated animals, mice, rats, non-human primates, and humans.

A “biological sample” encompasses any sample obtained from a cell, tissue, or organism. The definition encompasses blood and other liquid samples of biological origin, that are accessible from an organism through sampling by invasive means (e.g., surgery, open biopsy, endoscopic biopsy, and other procedures involving non-negligible risk) or by minimally invasive or non-invasive approaches (e.g., urine collection, blood drawing, needle aspiration, and other procedures involving minimal risk, discomfort or effort). The definition also includes samples that have been manipulated in any way after their procurement, such as by treatment with reagents, solubilization, or enrichment for certain components, such as proteins or organic metabolites. The term “biological sample” also encompasses a clinical sample such as serum, plasma, other biological fluid, or tissue samples, and also includes cells in culture, cell supernatants and cell lysates.

“Biological fluid” refers, but is not limited to, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, or other biological fluid.

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as M₀; for most organic molecules, this is the species containing all ¹²C, ¹H, ¹⁶O, ¹⁴N, etc. Other mass isotopomers are distinguished by their mass differences from M₀ (M₁, M₂, etc.). For a given mass isotopomer, the location or position of isotopes within the molecule is not specified and may vary (i.e., “positional isotopomers” are not distinguished).

“Mass isotopomer envelope” refers to the set of mass isotopomers comprising the family associated with each molecule or ion fragment monitored.

“Mass isotopomer pattern” refers to a histogram or plot of relative values of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonomously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m₀ and the masses of other mass isotopomers are identified by their mass differences from m₀ (m₁, m₂, etc.).

“Isotopically perturbed” refers to the state of an element or molecule that results from the explicit incorporation of an element or molecule with a distribution of isotopes that differs from the distribution that is most commonly found in nature, whether a naturally less abundant isotope is present in excess (enriched) or in deficit (depleted).

A “detectable amount” of an isotopic label is an amount that can be measured after incorporation into a biochemical component of an infectious organism, using any method suitable for quantitation of such isotopes, for example mass spectrometry or liquid scintillation counting.

“Monomer” refers to a chemical unit that combines during the synthesis of a polymer and which is present two or more times in the polymer.

“Polymer” refers to a molecule synthesized from and containing two or more repeats of a monomer.

“Protein” refers to a polymer of amino acids. As used herein, a “protein” may refer to long amino acid polymers as well as short polymers such as peptides.

“Sugar” refers to a monosaccharide, polysaccharide, or a monosaccharide and polysaccharide derivatives. Examples of sugar derivatives include, but are not limited to, glucoronic acid and glucosamine.

“Deuterated water” refers to water incorporating one or more ²H isotopes.

“Isolating” refers to separating one component from one or more additional components in a mixture of components. For example, isolating a biochemical component refers to separating one biochemical components from a mixture of biochemical components. Small quantities of additional biochemical components may be present in the isolated biochemical component.

Methods of the Invention

In one aspect, the invention provides methods for determining rates of replication (i.e., growth or proliferation) or destruction (i.e., death) of an infectious agent in an infected host organism. Methods of the invention include contacting with, or administering to the host organism a precursor molecule that contains a detectable amount of an isotopic label or labels, and allowing sufficient time for the incorporation of the label or labels into a biochemical component or components of the infectious agent residing in the host organism. The infectious agent, or the labeled biochemical component of the infectious agent, is isolated from a biological sample taken from the host. The isotopic content and/or pattern or the rate of change of the isotopic content and/or pattern in the biochemical component of the infectious agent is measured. An alternative method of the invention includes contacting with, or administering to the host organism a precursor molecule that contains a detectable amount of an isotopic label or labels, allowing sufficient time for incorporation of the label or labels into a biochemical component or components of the infectious agent residing in the host, then discontinuing administration or contacting of the precursor molecule to the host, and measuring over time the isotopic content and/or pattern or the rate of change of the isotopic content and/or pattern of the biochemical component isolated from the infectious agent. The rates of synthesis or breakdown of the component are then calculated and can be used to represent rates of replication (growth) or destruction (death) of the infectious agent.

A. Administration of an Isotope Labeled Precursor Molecule

1. Labeled Precursor Molecules

a. Isotope Labels

In the methods of the present invention, a detectable amount of isotopically-labeled precursor molecule is administered to, or contacted with, a host organism. An isotopic label may be either radioactive or non-radioactive. Examples of isotopes suitable for use as isotope labels include, but are not limited to, ¹³C, ¹⁴C, ²H, ³H, ¹⁵N, ³⁵S, ¹¹C, and ³⁵P.

In one embodiment, the isotope label is ²H.

b. Precursor Molecules

A labeled precursor molecule is capable of metabolic entry into the nutrient metabolic and biochemical pools of a host organism. A biochemical component of the infectious agent will become isotope-labeled when it utilizes isotopically labeled precursor molecules from the host metabolic pools for biosynthesis. In one embodiment, the entire isotope-labeled precursor molecule is incorporated into the biochemical component of the infectious agent. Alternatively, an isotope label containing portion of the isotope-labeled precursor molecule is incorporated into the biochemical component of the infectious agent.

The starting concentration of labeled precursor that will result in detectable label in an isolated microbe/microbial molecular component will depend on the turnover rate of the microbe and the degree of enrichment of the isotope. Hellerstein MK, Neese RA. Mass isotopomer distribution analysis at eight years: theoretical, analytic and experimental considerations. Am J Physiol 276 (Endocrinol Metab 39): E1146-E1162, 1999.

Administration of an isotopically-labeled precursor molecule to a host organism may be accomplished by a variety of methods that are well known in the art, including oral, parenteral, subcutaneous, intravascular (e.g., intravenous, intraarterial), intraperitoneal, intramuscular, intranasal, and intrathecal administration. The delivery may be systemic, regional, or local.

Administration of the isotope labeled precursor molecule may be continuous. Continuous administration for a selected duration may be accomplished, for example, by intravenous administration or by use of a controlled release carrier (e.g., an osmotic mini-pump). Alternatively, administration may be repeated at intervals. Administration may also be discontinuous. The precursor molecule may be formulated into appropriate forms for different routes of administration as described in the art, for example, in “Remington: The Science and Practice of Pharmacy,” Mack Publishing Company, Pennsylvania, 1995.

The isotopically labeled precursor molecule may be provided in a variety of formulations, including solutions, emulsions, suspensions, powders, tablets, and gels, and/or may be optionally incorporated in a controlled-release matrix. The formulations may include excipients available in the art, such as diluents, solvents, buffers, solubilizers, suspending agents, viscosity controlling agents, binders, lubricants, surfactants, preservatives, and stabilizers. The formulations may include bulking agents, chelating agents, and antioxidants. Where parenteral formulations are used, the formulation may additionally or alternately include sugars, amino acids, or electrolytes.

B. Obtaining One or More Biological Samples

One or more biological samples are obtained from an individual. The biological sample may be obtained by any method known in the art. Samples may be collected at a single time point or at multiple time points from one or more tissues or bodily fluids. The tissue or fluid may be collected using standard techniques in the art, such as, for example, tissue biopsy, blood draw, or collection of secretia or excretion from the body. Examples of suitable bodily fluids or tissues from which an infectious agent, or component thereof, may be isolated include urine, blood, intestinal fluid, edema fluid, saliva, lacrimal fluid (tears), inflammatory exudate, synovial fluid, abscess, empyema or other infected fluid, cerebrospinal fluid, pleural effusions, sweat, pulmonary secretions, seminal fluid, feces, bile, intestinal secretions, or any infected tissue including, but not limited to liver, intestinal epithelium, spleen, lung, pericardium, pleura, skin, muscle, synovium, cartilage, bone, bone marrow, thyroid gland, pancreas, brain, prostate, ovaries, endometrium, uterus, uterine cervix, testes, epididymis, bladder wall, kidney, adrenal, pituitary gland, adipose cells/tissue, omentum, or other cells and tissue.

The frequency of obtaining one or more biological samples can vary depending on different factors. Such factors include, but are not limited to, the nature of the biochemical component, ease and safety of sampling, biological rate constants and turnover kinetics of the precursor molecule or biochemical component, and the half-life of an agent administered to the host organism.

After administration of a labeled precursor molecule to an infected host, followed by a suitable time period for labeling of the infectious agent to occur, the infectious agent, or a biochemical component thereof, may be isolated from the biological sample.

The infectious agent may be isolated intact, or a biochemical component may be isolated, using separation procedures that are well known in the art. Typically, infectious agents that are isolated intact are subjected to further separation procedures to isolate one or more selected biochemical components of interest for quantitation of the contert and/or pattern of incorporated isotopic label. For example, intact microbes may be isolated from a bodily fluid using a technique such as density gradient centrifugation, immunoaffinity chromatography, or ultracentrifugation, and after appropriate treatment of the sample, selected components of the infectious agent may be isolated. For example, selected proteins may be isolated via techniques such as gel electrophoresis or isoelectric focusing. Other suitable isolation procedures for intact microbes and biochemical components are well known. See, for example, Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The one or more biochemical components of the infectious agents may also be purified partially purified, or optionally, isolated by conventional purification methods including high pressure liquid chromatography (HPLC), fast performance liquid chromatography (FPLC), chemical extraction, thin layer chromatography, gas chromatography, gel electrophoresis, and/or other separation methods known to those skilled in the art.

In another embodiment, the one or more biochemical components may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase or nuclease degradation). Hydrolysis or degradation may be conducted either before or after purification and/or isolation of the biochemical component. The biochemical components also may be partially purified, or optionally, isolated, by conventional purification methods including high performance liquid chromatography (HPLC), fast performance liquid chromatography (FPLC), gas chromatography, gel electrophoresis, and/or any other methods of separating chemical and/or biochemical compounds known to those skilled in the art.

In some circumstances, it is possible to measure incorporation of label (e.g., in a host fluid/tissue that does not contain interfering labeled molecules) without directly isolating the infectious agent or the labeled component. In such circumstances, excess label may be provided. Excess label is provided in the form of highly concentrated label or large quantities of labeled material.

### Measuring the Isotopic Content and/or Pattern of Incorporated Isotopic Label in Biochemical Components

The isotopic content of the isotopically labeled biochemical component of interest is measured to determine the isotopic content and/or pattern of label incorporated in the biochemical component (i.e., the relative proportion of isotopically labeled and unlabeled species of the biochemical component isolated). Isotopic content and/or pattern may be determined by various analytic methods known in the art, including mass spectrometry, liquid scintillation counting, PET scanning, NMR, near IR laser spectroscopy, laser-based detection, and geiger counter.

Incorporation of labeled isotopes into biological molecules may be measured directly. Alternatively, incorporation of labeled isotopes may be determined by measuring the incorporation of labeled isotopes into one or biochemical components, or hydrolysis or degradation products of biochemical components. The hydrolysis products may optionally be measured following either partial purification or isolation by any known separation method, as described previously.

Mass Spectrometry

Mass spectrometers convert components of a sample into rapidly moving gaseous ions and separate them on the basis of their mass-to-charge ratios. The distributions of isotopes or isotopologues of ions, or ion fragments, may thus be used to measure the isotopic enrichment in one or more biochemical components.

Generally, mass spectrometers include an ionization means and a mass analyzer. A number of different types of mass analyzers are known in the art. These include, but are not limited to, magnetic sector analyzers, electrostatic analyzers, quadrapoles, ion traps, time of flight mass analyzers, and fourier transform analyzers. In addition, two or more mass analyzers may be coupled (MS/MS) first to separate precursor ions, then to separate and measure gas phase fragment ions.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization. In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS is used to measure mass isotopomer abundances of molecules, hydrogen-labeled isotope incorporation from labeled water is amplified 3 to 7-fold, depending on the number of hydrogen atoms incorporated into the organic molecule from labeled water.

In one embodiment, isotope enrichments of biochemical components may be measured directly by mass spectrometry.

In another embodiment, the biochemical components may be partially purified, or optionally isolated, prior to mass spectral analysis. Furthermore, hydrolysis or degradation products of biochemical components may be purified.

### Calculation of Rate of Replication or Destruction of Infectious Agent

In one embodiment, a stable (i.e., non-radioactive) isotope-labeled precursor molecule is administered and the isotopic content and/or pattern of the biological catabolite or degradation product thereof is determined by mass spectrometry, using techniques that are well known in the art (see, e.g., U.S. Pat. Nos. 5,388,686, 5,910,403, and 6,010,846, previously incorporated by reference). Often, the isotopic content is expressed as fractional abundance of mass isotopomers of a biomolecule of interest. As used herein, “fractional abundance” refers to the fraction of the total abundance of a particular isotope or mass isotopomer. For a mass isotopomer M_{X}, Fractional abundance of

$${M}_{x} = {A}_{x} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

Δ A Fractional abundance (enrichment or depletion)=

$${( {A}_{x} )}_{e} - {( {A}_{x} )}_{b} = {( \frac{Abundance {M}_{x}}{{\sum }_{i = 0}^{n} Abundance {M}_{i}} )}_{e} - {( \frac{Abundance {M}_{x}}{{\sum }_{i = 0}^{n} Abundance {M}_{i}} )}_{b} ,$$

where subscript e refers to enriched and b refers to baseline or natural abundance.

The content and/or pattern of incorporated labeled precursor molecule, determined as described above, may be used to calculate a rate of replication or destruction of an infectious agent within a host organism. For example, the rate of synthesis, breakdown, or turnover of a microbial biochemical component may be calculated by application of formulae that are well known in the art, such as the precursor-product equation, isotope dilution equations, or calculation of other kinetic parameters of interest. See, for example, (i) Wolfe, R. R. 1984. Tracers in Metabolic Research. Radio-Isotope and Stable Isotope/Mass Spectrometric Methods. Alan R. Liss, Inc., NY.) (ii) Hellerstein MK, Neese R. Mass isotopomer distribution analysis: a technique for measuring biosynthesis and turnover of polymers. Am J Physiol 263:E988-E1001, 1992. (iii) Hellerstein M K, Neese R A. Mass isotopomer distribution analysis at eight years: theoretical, analytic and experimental considerations. Am J Physiol 276 (Endocrinol Metab 39): E1146-E1162, 1999. (iv) Zilversmit, D. B., C. Entenman, and M. Fishler. 1943. The calculation of turnover rate and turnover time from experiments involving the use of labeling agents. J Gen. Physiol. 26:325-331. (v) Hellerstein M. Methods for measuring polymerisation biosynthesis: three general solutions to the problem of the “true precursor.” Diabetes Nutr Metab 13(1):46-60, 2000.

In addition to the above-cited references, calculation software implementing the method is publicly available from Professor Marc Hellerstein, University of Calif., Berkeley.

### Determining the rate of Replication or Destruction ofan Infectious Agent

In one aspect, methods of the invention are used to calculate the rate at which a population of infectious agents replicates in an infected host organism. An isotopically-labeled precursor molecule is administered to the host organism and after a selected period of time, the isotopic content and/or pattern of label incorporated into a biochemical component of the infectious agent is determined, as described above. Typically, sampling (i.e., removal of a tissue or body fluid sample from the host) is performed while the labeled precursor molecule is being administered. The isotopic content and/or pattern of label incorporated over time reflects and reveals the rate of synthesis of the component and thus reveals the rate of replication of the infectious agent in vivo to practitioners skilled in the art. The isotopic content of a labeled precursor molecule that will result in detectable label in an isolated biochemical component will depend on the turnover rate of the infectious agent and factors influencing dilution of the labeled biosynthetic precursor that is administered in the nutrient metabolic or biochemical pools of the host. Hellerstein M K, Neese R A. Mass isotopomer distribution analysis at eight years: theoretical, analytic and experimental considerations. Am J Physiol 276 (Endocrinol Metab 39): E1146-E1162, 1999.

In another aspect, methods of the invention are used to calculate the rate of destruction of an infectious agent in a host organism. An isotopically-labeled precursor molecule is administered to the host organism, thereby isotopically enriching one or more biochemical components of the infectious agent as described above. After cessation of label. administration, samples are taken from the host at sequential time points and isotopic content and/or pattern in a biochemical component of the infectious agent is determined at these time points. The rate of breakdown or isotopic decay of the biochemical component over time reveals the rate of destruction (or removal rate) of the infectious organism in vivo to practitioners skilled in the art.

Methods of Use

The antimicrobial or immunostimulatory effect of a drug agent may be tested using the methods described herein. A decrease in the rate of replication of an infectious agent or an increase in the rate of destruction of an infectious agent indicates that the agent has an antimicrobial or immunostimulatory effect.

Drug agents may be any chemical compound or composition known in the art. Drug agents include, but are not limited to, any chemical compound or composition disclosed in, for example, the 13th Edition of The Merck Index (a U.S. publication, Whitehouse Station, N.J., USA), incorporated herein by reference in its entirety.

Kits of the Invention

The invention provides kits for carrying out the methods of the invention. Kits of the invention include reagents for use in the methods described herein, in one or more containers. Kits may include isotopically labeled precursor molecules, buffers, and/or excipients, separately or in combination. Each reagent is supplied in a solid form or liquid buffer that is suitable for inventory storage, and later for exchange into a medium suitable for administration to a host organism in accordance with methods of the invention. Kits may also include means for administering the labeled precursor molecules and/or means for obtaining a sample of a tissue or biological fluid from the host organism.

Kits of the invention are provided in suitable packaging. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like.

Kits of the invention may optionally include a set of instructions in printed or electronic (e.g., magnetic or optical disk) form, relating information regarding the components of the kits and their administration to a host organism and/or how to measure label incorporated into a biochemical component of an infectious agent. The kit may also be commercialized as part of a larger package that includes instrumentation for measuring isotopic content of a biochemical component, such as, for example, a mass spectrometer.

Isotopically Labeled Infections Agents, Precursor Molecules, and Biochemical Components

Isotope labeled infectious agents, isotope labeled precursor molecules, and isotope labeled biochemical components made by the methods described herein are independently useful. Such labeled infectious agents and precursor molecules can be injected into a host organism or test subject and used as a reagent to study turnover of the reagent in vivo. Furthermore, such microbes and macromolecules can be utilized in in vitro studies to determine the effects of inhibitors on their synthesis or degradation.

### EXAMPLES

The following non-limiting examples further illustrate the methods and compositions disclosed herein:

### Example 1

In an illustrative embodiment of the methods of the invention, an isotopically-labeled amino acid such as leucine (e.g., [¹³C]-leucine, [²H]-leucine, [¹⁴C]-leucine) is administered by constant intravenous infusion to a human being who is known to be infected with the human immunodeficiency virus and who is under treatment with an anti-retroviral agent. Blood is removed from the subject during and after completion of the infusion of labeled leucine and the human immunodeficiency virus is isolated from the blood plasma by ultracentrifugation. Gel electrophoresis is then performed to isolate specific proteins contained in the virus (e.g., the Gag protein products, P₂₄, P₁₇, and P₇). These proteins are then hydrolyzed in acid to free leucine, which is analyzed by mass spectrometry. The rate of rise of isotopically-labeled leucine in the leucine isolated from the Gag protein products during the infusion of isotopically-labeled leucine reveals the synthesis or replication rate of virus in the blood compartment. The rate of decay of isotopically labeled leucine in the leucine isolated from the Gag protein products after completion of the infusion of isotopically-labeled leucine reveals the removal or destruction rate of virus from the blood compartment. The effect of different anti-retroviral therapies on these kinetic parameters can be used as an index of drug efficacy in an individual or a group of patients.

### Example 2

Isotopically-labeled water is administered orally to a human being who is known to be infected with the human immunodeficiency virus and who is under treatment with an anti-retroviral agent. In one such embodiment, this is ²H₂O (at a dose of 50 ml, for example) given orally to drink with morning and evening meals for 42 days (6 weeks). A blood or urine aliquot (10 ml) is collected from the individual at a defined time point or points (e.g., on the final day of the ²H₂O protocol (day 42)).

Blood is removed from the subject during and after completion of isotope labeled water administration and the human immunodeficiency virus is isolated from the blood plasma by ultracentrifugation. Gel electrophoresis is then performed to isolate specific proteins contained in the virus (e.g., the Gag protein products, P₂₄, P₁₇, and P₇). These proteins are then hydrolyzed in acid to free amino acids, which are analyzed by mass spectrometry.

The rate of rise of isotopically-labeled amino acids, for example, isotopically-labeled alanine, glycine or other non-essential amino acid, isolated from the Gag protein products during the administration of labeled water reveals the synthesis or replication rate of virus in the blood compartment. The rate of decay of isotopically labeled amino acid in the amino acid isolated from the Gag protein products after completion of the administration of labeled water reveals the removal or destruction rate of virus from the blood compartment. The effect of different anti-retroviral therapies on these kinetic parameters can be used as an index of drug efficacy in an individual or a group of patients. By administering labeled water to label other HIV biochemical components such as RNA, the changes in the rate of synthesis or degradation of these isotopically-labeled biochemical components (e.g., HIV RNA) can also be used to measure the replication or destruction of HIV virions. Techniques for labeling and measuring nucleic acids (e.g., RNA) are described in U.S. Pat. Nos. 6,461,806, 6,010,846, and 5,910,403, previously incorporated by reference.

### Example 3

Isotopically-labeled water is administered orally to a human being who is known to be infected with Mycobacterium tuberculosis and who is under treatment with an antibiotic or immune stimulant. In one such embodiment, this is ²H₂O (at a dose of 50 ml, for example) given orally to drink with morning and evening meals for 42 days (6 weeks). A blood or urine aliquot (10 ml) is collected from the individual at a defined time point or points (e.g., on the final day of the ²H₂O protocol (day 42).

A sputum sample or lung biopsy is taken from the subject during and after completion of administration of labeled water and the Mycobacterium tuberculosis is isolated by techniques known in the art. Alternatively, M. tuberculosis DNA is isolated from the sample taken from the host organism without isolating the M. tuberculosis from the sample. The total DNA is then isolated. The DNA is hydrolyzed into nucleosides, which are analyzed by mass spectrometry. Alternatively, isolated nucleosides are derivatized for gas chromatographic/mass spectrometric analysis, according to known methods. By one such method, the trimethylsilyl (TMS) derivative of deoxycytosine and other nucleosides present is formed with bis(trimethyl[silyl])acetamide.

The rate of incorporation of isotopically-labeled nucleosides into DNA reveals the synthesis or replication rate of Mycobacterium tuberculosis in the individual. The rate of decay of isotopically labeled nucleosides after completion of administering isotopically labeled water reveals the removal or destruction rate of Mycobacterium tuberculosis from the individual. The effect of different antibiotic and immunostimulant therapies on these kinetic parameters can be used as an index of drug efficacy in an individual or a group of patients.

### Example 4

Isotopically-labeled water is administered orally to a human being who is known to be infected with Plasmodium falcipurum. In one such embodiment, this is 2H₂O (at a dose of 50 ml, for example) given orally to drink with morning and evening meals for 42 days (6 weeks). A blood or urine aliquot (10 ml) is collected from the individual at a defined time point or points (e.g., on the final day of the ²H₂O protocol (day 42).

A blood sample is removed from the subject during and after the administration of labeled water, and the red blood cells are isolated by methods known in the art. Nuclear DNA in the red blood cells is then isolated by conventional methods. Red blood cells do not contain any DNA, so DNA detected by the methods herein is from Plasmodium falcipurum. The DNA is then hydrolyzed in acid to free nucleosides, which are analyzed by mass spectrometry. Optionally, isolated nucleosides are derivatized for gas chromatographic/mass spectrometric analysis, according to known methods. By one such method, the trimethylsilyl (TMS) derivative of deoxycytosine and other nucleosides present is formed with bis(trimethyl[silyl])acetamide.

The rate of incorporation of isotopically labeled nucleosides into DNA during administration of isotopically labeled water reveals the replication rate of the Plasmodium falcipurum in the host organism. The rate of decay of isotopically labeled nucleosides after completion of administering isotopically labeled water reveals the removal or destruction rate of the Plasmodium falcipurum in the host organism.

The effect of different biological and therapeutic agents on these kinetic parameters can be used as an index of drug efficacy in an individual or a group of patients.

### Example 5

Isotopically-labeled water is administered orally to a human being who is known to be infected with Streptococcus pneumonia. In one such embodiment, this is ²H₂O (at a dose of 50 ml, for example) given orally to drink with morning and evening meals for 42 days (6 weeks). A blood or urine aliquot (10 ml) is collected from the individual at a defined time point or points (e.g., on the final day of the ²H₂O protocol (day 42).

A bodily sample from a lung abscess (e.g., aspirated infected fluid) is obtained from the subject during and after the administration of labeled water and the S. pneumonia DNA is then isolated by conventional methods known in the art (e.g., complementary hybridization). Alternatively, the S. pneumonia infectious agent is isolated from the host organism (e.g., from the aspirated infected fluid) and the S. pneumonia DNA is then isolated from the infectious agent. The DNA is then hydrolyzed in acid to free nucleosides, which are analyzed by mass spectrometry. Optionally, isolated nucleosides are derivatized for gas chromatographic/mass spectrometric analysis, according to known methods. By one such method, the trimethylsilyl (TMS) derivative of deoxycytosine and other nucleosides present is formed with bis(trimethyl[silyl])acetamide.

The rate of incorporation of isotopically labeled nucleosides into DNA during administration of isotopically labeled water reveals the replication rate of the Streptococcus pneumonia in the host organism. The rate of decay of DNA as measured by the rate of decay of isotopically labeled nucleosides after completion of administering isotopically labeled water reveals the removal or destruction rate of the Streptococcus pneumonia in the host organism.

The effect of known or potential antibiotic or immunostimulatory agents on these kinetic parameters can be used as an index of drug efficacy in an individual or a group of patients.

Although the foregoing invention has been described in some detail by way of illustration and examples for purposes of clarity of understanding, it will be apparent to those skilled in the art that certain changes and modifications may be practiced without departing from the spirit and scope of the invention.

All publications, patents, and patent applications cited herein are hereby incorporated by reference in their entirety for all purposes to the same extent as if each individual publication, patent, or patent application were specifically and individually indicated to be so incorporated by reference.

1. A method of determining the rate of replication or destruction of an infectious agent in a host organism, said method comprising:
(a) administering an isotope-labeled precursor molecule to said host organism for a period of time sufficient for the isotope label of said isotope-labeled precursor molecule to become incorporated into a biochemical component of said infectious agent;
(b) obtaining one or more biological samples from the host organism, wherein said one or more biological samples comprise the infectious agent or said biochemical component of said infectious agent;
(c) measuring the isotopic content, rate of change of isotopic content, pattern or rate of change of pattern of said isotopic content in said biochemical component; and
(d) calculating the rate of synthesis or breakdown of the biochemical component to determine the rate of replication or destruction of said infectious agent in said host organism.

2. The method of claim 1, wherein said biological sample is a tissue of the host organism.

3. The method of claim 1, wherein said biological sample is a bodily fluid of the host organism.

4. The method of claim 1, wherein said host organism is a mammal.

5. The method of claim 4, wherein said mammal is a human.

6. The method of claim 1, wherein said infectious agent is selected from the group consisting of bacteria, viruses, protozoa, yeast, and parasites.

7. The method of claim 1, wherein said infectious agent is human immunodeficiency virus, hepatitis B or C virus, or other clinically important virus.

8. The method of claim 1, wherein said biochemical component is selected from the group consisting of DNA, RNA, proteins, lipids, carbohydrates, and porphyrins.

9. The method of claim 1, wherein said isotopic label is selected from the group consisting of ¹³C, ¹⁴C, ²H, ³H, ¹⁵N, ³⁵S, ¹¹C, and ³⁵P.

10. The method of claim 9, wherein said isotopic label is ²H.

11. The method of claim 1, further comprising collecting a plurality of biological samples from said host organism.

12. The method of claim 1, wherein measurement of the isotopic content and/or pattern or the rate of change of isotopic content and/or pattern in the biochemical component is performed by mass spectrometry.

13. The method of claim 3 wherein the fluid is selected from the group consisting of urine, blood, saliva, interstitial fluid, edema fluid, lacrimal fluid, inflammatory exudates, synovial fluid, abcess, empyema, cerebrospinal fluid, sweat, pulmonary secretions, seminal fluid, feces, bile, and intestinal secretions.

14. A method of identifying an antimicrobial or immunostimulatory effect of a drug agent, comprising:
a) determining the rate of replication or destruction of an infectious agent in a host organism according to claim 1;
b) administering the drug agent to said host organism; and
c) determining the rate of replication or destruction of the infectious agent in a host organism according to claim 1, wherein a decrease in the rate of replication or an increase in the rate of destruction of the infectious agent indicates an antimicrobial or immunostimulatory effect of the drug agent.

15. The method of claim 14, wherein the effect of said antimicrobial or immunostimulatory agent on the growth or death of the infectious agent in the host organism is used as a diagnostic test in clinical patient care or as a biomarker tool for drug discovery, development, or approval of an antimicrobial or immunostimulatory agent.

16. A method of identifying an antimicrobial or immunostimulatory effect of a drug agent, comprising:
a) determining the rate of replication or destruction of an infectious agent in a first host organism according to claim 1, wherein the drug agent has not been administered to said first host organism;
b) determining the rate of replication or destruction of an infectious agent in a second host organism according to claim 1, wherein the drug agent has been administered to said second host organism;
c) comparing the rate of replication or destruction of the infectious agent in said first and second host organisms, wherein a lower value for in the rate of replication or an increase in the rate of destruction of the infectious agent in the second host organism indicates an antimicrobial or immunostimulatory effect of the drug agent.

17. The method of claim 16, wherein the effect of said antimicrobial or immunostimulatory agent on the growth or death of the infectious agent in the host organism is used as a diagnostic test in clinical patient care or as a biomarker tool for drug discovery, development, or approval of an antimicrobial or immunostimulatory agent.

18. A kit for determining the rate of replication or destruction of an infectious agent in a host organism comprising
a) an isotope-labeled precursor molecule, and
b) instructions for use of the kit to determine the rate of replication or destruction of the infectious organism.

19. The kit of claim 18, further comprising a tool for administration of precursor molecules.

20. The kit of claim 18, further comprising an instrument for collecting a sample from a host organism.

21-22. (canceled)

23. An isolated infectious agent comprising an isotope labeled precursor molecule.

24. An isolated infectious agent comprising an isotope labeled biochemical component.

25. (canceled)

26. An isolated isotope-labeled biochemical component obtained from an infectious agent.

27. An isolated isotope-labeled biochemical component made by administering an isotope-labeled precursor molecule to a host organism for a period of time sufficient for the isotope label of said isotope-labeled precursor molecule to become incorporated into a biochemical component of an infectious agent in said host organism to produce the isotope-labeled biochemical component.

28. (canceled)
